(19) United States
(12) Patent Application Publication
(10) Pub. No.: US 2011/0070314 A1
(43) Pub. Date: Mar. 24, 2011

(54) METHODS FOR TREATING BRAIN TUMORS

(76) Inventors: Yong Joon Jo, Closter, NJ (US); Yong Jin Yang, (US)

(21) Appl. No.: 12/879,316

(22) Filed: Sep. 10, 2010

Related U.S. Application Data
(60) Provisional application No. 61/243,648, filed on Sep. 18, 2009.

(51) Int. Cl. A61K 33/36; A61P 35/00

(52) U.S. Cl. 424/623

(57) ABSTRACT

The present invention relates to methods treating brain tumors comprising administering a subject in need thereof a therapeutically effective amount of sodium meta arsenite, alone or in combination with another anti-brain tumor medicament.

### CROSS-REFERENCE OF RELATED APPLICATION

This application claims priority to provisional application Ser. No. 61/243,648, filed Sep. 18, 2009.

### FIELD OF THE INVENTION

The present invention provides methods for treating brain tumors such as glioma, medulloblastoma or meningioma; more particularly the present invention provides a prophylactically and/or therapeutically effective amount or regimen of sodium meta arsenite for preventing, treating, reducing, or eliminating brain tumors.

### BACKGROUND OF THE INVENTION

The blood-brain barrier (BBB) is a transvascular permeability barrier that tightly controls entry of substances into the brain. Unlike capillaries that serve other areas of the body, the capillaries that perfuse the brain are lined with special endothelial cells that lack fenestrations and are sealed by endothelial tight junctions. This tight endothelium provides a physical barrier that together with metabolic barriers is thought to form the basis of the BBB.

The BBB protects the brain against pathogens (e.g., viruses) and other dangers of the circulatory system, including changes in the composition of the systemic blood supply (e.g., electrolyte levels). The barrier is not complete, however, and permits entry of certain substances, such as small fat-soluble (lipophilic) molecules that can freely diffuse through the barrier. The BBB also permits entry of essential nutrients, such as glucose and amino acids, which are vital to brain function. These nutrients are generally water soluble (hydrophilic), and require more complex mechanisms for crossing the BBB, such as carrier-mediated transport, receptor-mediated transcytosis and absorptive-mediated transcytosis.

While protective under normal circumstances, the BBB frustrates delivery of drugs and other therapeutic molecules to the brain. It has been reported that the BBB blocks delivery of more than 98% of central nervous system (CNS) drugs (Pardridge, W J. “Nature Rev.: Drug Discovery 2002 1:131-139). The drug delivery challenge posed by the BBB is compelling, particularly as the population ages and the incidence of neurodegenerative diseases such as stroke, Alzheimer's disease, and Parkinson's disease increase in prevalence. The problem is particularly acute for patients with malignant brain tumors, who cannot benefit from anticancer drugs effective in treating tumors elsewhere in the body. Thus, there remains a significant need in the art for an anticancer drug that can cross the blood brain barrier and effectively treat brain tumors.

### SUMMARY OF THE INVENTION

According to the present invention, the treatment of brain tumors is achieved by administering to a subject in need thereof a therapeutically effective amount of sodium meta arsenite (NaAsO2) alone or in combination with other anticancer medicaments or therapies. Other non-limiting examples of anticancer medicaments include alkylating, agents, antifolates and topoisomerases and if appropriate, chemosensitizing agents.

In one aspect of the present invention, the invention relates to a method of treating brain tumors comprising administering a subject in need thereof a therapeutically effective amount of sodium meta arsenite. In one embodiment related to this aspect of the invention, the brain tumor is glioma. In another related embodiment, sodium meta arsenite is administered orally or by injection. In yet another related embodiment, sodium meta arsenite is administered in a unit dose of 0.001 mg to 20 mg/kg, once or more per day and in certain embodiments, in a dose of 0.5 mg/Kg per day.

### BRIEF DESCRIPTION OF THE DRAWINGS

FIGS. 1A-1D are graphs showing the effects of sodium meta arsenite on glioblastoma cell viability in vivo. (A) U-87MG cells, (B) U373 cells, (C) T98G cells, (D) U138 cells. Values are expressed as mean±S.D.

FIG. 2A-2D are graphs showing the effects of sodium meta arsenite on glioblastoma cell viability. (A) U-87MG, (B) U373, (C) T98G, (D) U138. Values are expressed as mean±S.D.

FIG. 3. Comparison of tumor volume of between control and sodium meta arsenite treated groups. (A) MRI image, (B) Tumor mass volume (Values are expressed as mean±S.D.), and (C) body weight.

FIG. 4. Comparison of tumor volume of between control and sodium meta arsenite treated groups. (A) Tumor mass volume (Values are expressed as mean±S.D.), (B) AST, ALT activities.

### DETAILED DESCRIPTION OF THE INVENTION

The present invention provides methods for preventing, treating, and/or managing brain tumors, the method comprising administering to a subject in need thereof a therapeutically effective amount of sodium meta arsenite (SMA) that reduces or eliminates brain tumor(s).

This invention is in part based on findings that sodium meta arsenite, a drug in phase I/II clinical trials for prostate cancer, can readily cross the blood brain barrier. It has also been found, in both in-vitro and in-vivo studies, that sodium meta arsenite can negatively affect brain tumor cells and brain tumors in mice and humans. Thus, the use of sodium meta arsenite to treat brain tumors eliminates the need to use osmotic blood brain barrier disruption, which is commonly used with chemotherapy agents to treat brain tumors, since most drugs that are effective in the treatment of brain tumors do not penetrate the blood brain barrier.

It has also been found that sodium meta arsenite is an effective treatment for brain tumors that are resistant to other medicaments.

### DEFINITIONS

As used herein, the term “brain tumor” refers to all neoplastic cell growth and proliferation, whether malignant or benign, and all pre-cancerous and cancerous cells and tissues of brain.

As used herein, the term “therapeutically effective amount” refers to an amount of sodium meta arsenite that is sufficient to prevent the development, recurrence, or onset of brain tumors or a symptom thereof; to enhance or improve the prophylactic effect(s) of another brain tumor therapy; to reduce the severity and duration of brain tumors; to ameliorate one or more symptoms of brain tumors; to prevent the advancement of brain tumors; to cause regression of brain tumors; and/or to enhance or improve the therapeutic effect(s) of another therapy for brain tumors. In an embodiment of the invention, the therapeutically effective amount of sodium meta arsenite is an amount that is effective to achieve one, two or three or more of the following results once it is administered: (1) a reduction or elimination of the brain tumor; (2) a reduction in the growth of a brain tumor; (3) an impairment in the formation of a brain tumor; (4) eradication, removal, or control of primary, regional and/or metastatic brain cancer; (5) an increase in disease-free, relapse-free, progression-free, and/or overall survival of a subject with brain tumor; (6) an increase in the response rate, the durability of response, or number of brain tumor patients who respond or are in remission; (7) the size of the brain tumor is maintained and does not increase or increases by less than 10%, or less than 5%, or less than 4%, or less than 2%, (8) an increase in the number of brain tumor patients in remission, (9) an increase in the length or duration of remission, (10) a decrease in the recurrence rate of brain tumor, (11) an increase in the time to recurrence of brain tumor, and (12) an amelioration of brain tumor-related symptoms and/or quality of life.

As used herein, the term “therapeutically effective regimen” refers to a regimen for dosing, timing, frequency, and duration of the administration of sodium meta arsenite for the treatment and/or management of brain tumors or a symptom thereof. In a specific embodiment, the regimen achieves one, two, three, or more of the following results: (1) a reduction or elimination of the brain tumor; (2) a reduction in the growth of a brain tumor; (3) an impairment in the formation of a brain tumor; (4) eradication, removal, or control of primary, regional and/or metastatic brain cancer; (5) an increase in disease-free, relapse-free, progression-free, and/or overall survival of a subject with brain tumor; (6) an increase in the response rate, the durability of response, or number of brain tumor patients who respond or are in remission; (7) the size of the brain tumor is maintained and does not increase or increases by less than 10%, or less than 5%, or less than 4%, or less than 2%, (8) an increase in the number of brain tumor patients in remission, (9) an increase in the length or duration of remission, (10) a decrease in the recurrence rate of brain tumor, (11) an increase in the time to recurrence of brain tumor, and (12) an amelioration of brain tumor-related symptoms and/or quality of life.

As used herein, the terms “subject” and “patient” are used interchangeably. As used herein, the term “subject” refers to an animal, preferably a mammal such as a non-primate (e.g., cows, pigs, horses, cats, dogs, rats, etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human, e.g., at least seventy years old. In another embodiment, the subject is a human adult, i.e., twenty-one years or older. In another embodiment, the subject is a human child. In yet another embodiment, the subject is a human infant.

### 1. Brain Tumors

The present methods are applicable to the treatment of brain tumors, such as glioblastoma. In general, the goals of brain tumor treatments are to remove as many tumor cells as possible, e.g., with surgery, kill as many of the cells left behind after surgery as possible with radiation and/or chemotherapy, and put remaining tumor cells into a nondividing, quiescent, non-invasive state for as long as possible with radiation and chemotherapy. Careful imaging surveillance is a crucial part of medical care, because tumor regrowth requires alteration of current treatment, or, for patients in the observation phase, restarting treatment.

Brain tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade II astocytomas are the most indolent of the diffuse astrocytoma spectrum. Astrocytomas display a remarkable tendency to infiltrate the surrounding brain, confounding therapeutic attempts at local control. These invasive abilities are often apparent in low-grade as well as high-grade tumors.

Glioblastoma multiforme is the most malignant stage of astrocytoma, with survival times of less than 2 years for most patients. Histologically, these tumors are characterized by dense cellularity, high proliferation indices, endothelial proliferation and focal necrosis. The highly proliferative nature of these lesions likely results from multiple mitogenic effects. One of the hallmarks of GBM is endothelial proliferation.

There are biologic subsets of astrocytomas, which may reflect the clinical heterogeneity observed in these tumors. These subsets include brain stem gliomas, which are a form of pediatric diffuse, fibrillary astrocytoma that often follow a malignant course. Brain stem GBMs share genetic features with those adult GBMs that affect younger patients. Pleomorphic xanthoastrocytoma (PXA) is a superficial, low-grade astrocytic tumor that predominantly affects young adults. While these tumors have a bizarre histological appearance, they are typically slow-growing tumors that may be amenable to surgical cure. Some PXAs, however, may recur as GBM. Pilocytic astrocytoma is the most common astrocytic tumor of childhood and differs clinically and histopathologically from the diffuse, fibrillary astrocytoma that affects adults. Pilocytic astrocytomas do not have the same genomic alterations as diffuse, fibrillary astrocytomas. Subependymal giant cell astrocytomas (SEGA) are periventricular, low-grade astrocytic tumors that are usually associated with tuberous sclerosis (TS), and are histologically identical to the so-called “candle-gutterings” that line the ventricles of TS patients. Similar to the other tumorous lesions in TS, these are slowly-growing and may be more akin to hamartomas than true neoplasms. Desmoplastic cerebral astrocytoma of infancy (DCAI) and desmoplastic infantile ganglioglioma (DIGG) are large, superficial, usually cystic, benign astrocytomas that affect children in the first year or two of life.

Oligodendrogliomas and oligoastrocytomas (mixed gliomas) are diffuse, usually cerebral tumors that are clinically and biologically most closely related to the diffuse, fibrillary astrocytomas. The tumors, however, are far less common than astrocytomas and have generally better prognoses than the diffuse astrocytomas. Oligodendrogliomas and oligoastrocytomas may progress, either to WHO grade III anaplastic oligodendroglioma or anaplastic oligoastrocytoma, or to WHO grade IV GBM. Thus, the genetic changes that lead to oligodendroglial tumors constitute yet another pathway to GBM.

Ependymomas are a clinically diverse group of gliomas that vary from aggressive intraventricular tumors of children to benign spinal cord tumors in adults. Transitions of ependymoma to GBM are rare. Choroid plexus tumors are also a varied group of tumors that preferentially occur in the ventricular system, ranging from aggressive supratentorial intraventricular tumors of children to benign cerebellopontine angle tumors of adults. Choroid plexus tumors have been reported occasionally in patients with Li-Fraumeni syndrome and von Hippel-Lindau (VHL) disease.

Medulloblastomas are highly malignant, primitive tumors that arise in the posterior fossa, primarily in children. Meningiomas are common intracranial tumors that arise in the meninges and compress the underlying brain. Meningiomas are usually benign, but some “atypical” meningiomas may recur locally, and some meningiomas are frankly malignant and may invade the brain or metastasize. Atypical and malignant meningiomas are not as common as benign meningiomas. Schwannomas are benign tumors that arise on peripheral nerves. Schwannomas may arise on cranial nerves, particularly the vestibular portion of the eighth cranial nerve (vestibular schwannomas, acoustic neuromas) where they present as cerebellopontine angle masses. Hemangioblastomas are tumors of uncertain origin that are composed of endothelial cells, pericytes and so-called stromal cells. These benign tumors most frequently occur in the cerebellum and spinal cord of young adults. Multiple hemangioblastomas are characteristic of von Hippel-Lindau disease (VHL). Hemangiopericytomas (HPCs) are dural tumors which may display locally aggressive behavior and may metastasize. The histogenesis of dural-based hemangiopericytoma (HPC) has long been debated, with some authors classifying it as a distinct entity and others classifying it as a subtype of meningioma.

The symptoms of both primary and metastatic brain tumors depend mainly on the location in the brain and the size of the tumor. Since each area of the brain is responsible for specific functions, the symptoms will vary a great deal. Tumors in the frontal lobe of the brain may cause weakness and paralysis, mood disturbances, difficulty thinking, confusion and disorientation, and wide emotional mood swings. Parietal lobe tumors may cause seizures, numbness or paralysis, difficulty with handwriting, inability to perform simple mathematical problems, difficulty with certain movements, and loss of the sense of touch. Tumors in the occipital lobe can cause loss of vision in half of each visual field, visual hallucinations, and seizures. Temporal lobe tumors can cause seizures, perceptual and spatial disturbances, and receptive aphasia. If a tumor occurs in the cerebellum, the person may have ataxia, loss of coordination, headaches, and vomiting. Tumors in the hypothalamus may cause emotional changes, and changes in the perception of hot and cold. In addition, hypothalamic tumors may affect growth and nutrition in children. With the exception of the cerebellum, a tumor on one side of the brain causes symptoms and impairment on the opposite side of the body.

### 2. Prophylactic & Therapeutic Uses of Sodium Meta Arsenite

The invention provides a method for preventing, treating, and/or managing a brain tumor, the method comprising administering to a subject in need thereof a prophylactically or therapeutically effective amount or regimen of sodium meta arsenite, wherein the amount or regimen results in at least an approximately 5% reduction in size of the brain tumor. In certain embodiments, the reduction in size of the brain tumor is monitored periodically. Accordingly, in a specific embodiment, the invention provides a method of preventing, treating and/or managing brain tumor in a subject, the method comprising: (a). administering to a subject in need thereof one or more doses of an effective amount of sodium meta arsenite; (b). monitoring the brain tumor in the subject prior to, during, and/or after administration of a certain number of doses and prior to the administration of a subsequent dose; and (c). detecting at least a 5% reduction in size of the brain tumor in the subject by repeating step (a) as necessary.

In certain embodiments, the amount or regimen of sodium meta arsenite results in at least a 5%, 10%, 15%, 20%, 30%, 40%, 50%, 60%, 70%, 80%, 90%, 95%, 98% or 99% reduction in size of the brain tumor. For example, in some embodiments, the amount or regimen of sodium meta arsenite results in at least an approximately 5%-99%, a 5%-80%, a 5 to 40%, a 10% to 99%, a 10 to 80%, a 10-60%, a 10%-40%, a 20 to 99%, a 20%-80%, a 20%-60%, a 20%-40%, a 50%-98%, 50%-80%, or a 60%-99% reduction in size of the brain.

In other embodiments, the amount or regimen of sodium meta arsenite results in at least a 1.1-, 1.2-1.5-, 2-, 3-, 4-, 5-, 10-, 25-, 50-, 75-, 100-, 200- or 1000-fold reduction in size of the brain tumor. In some embodiments, the reduction in size of the brain tumor results after two weeks, a month, two months, three months, four months, six months, nine months, 1 year, 2 years, 3 years, or 4 years of administration of the regimen.

In some embodiments, the amount or regimen of sodium meta arsenite results in a reduction in the bulk brain tumor size as well as a reduction in the brain cancer cells. In certain embodiments, the reduction in the bulk brain tumor size and the reduction in the brain cancer cells are monitored periodically. Accordingly, in one embodiment, the invention provides a method of preventing, treating and/or managing brain tumor in a subject, the method comprising: (a) administering to a subject in need thereof one or more doses of an effective amount of sodium meta arsenite; (b) monitoring the brain cells and the bulk brain tumor size in the subject prior to, during, and/or after administration of a certain number of doses and prior to the administration of a subsequent dose; and (c) detecting at least a 5% reduction in the amount of brain cancer cells and/or the bulk brain tumor size in the subject by repeating step (a) as necessary.

In certain embodiments, the amount or regimen of sodium meta arsenite results in at least an approximately 5%, 10%, 15%, 20%, 25%, 30%, 40%, 50%, 60%, 70%, 75%, 80%, 90%, 95%, 98% or 99% reduction in the brain cancer cells and the bulk brain tumor size. For example, in some embodiments, the regimen results in an approximately 2%-98%, a 5%-80%, a 5 to 40%, a 10% to 99%, a 10 to 80%, a 10-60%, a 10%-40%, a 20 to 99%, a 20%-80%, a 20%-60%, a 20%-40%, a 50%-99%, 50%-80%, or a 60%-99% reduction in the brain cancer cells and the bulk brain tumor size. In other specific embodiments, the regimen results in at least a 1.1-, 1.2-1.5-, 2-, 2.5-, 3-, 4-, 5-, 10-, 20-, 25-, 50-, 75-, 100-, 200-, or 1000-fold reduction in the amount of brain cancer cells and/or the bulk brain tumor size. In some embodiments, the reductions in the brain cancer cells and the bulk brain tumor size result after two weeks, a month, two months, three months, four months, six months, nine months, 1 year, 2 years, 3 years, 4 years, 5 years or 10 years of administration of the regimen.

A number of known methods can be used to assess the bulk size of the tumor. Non-limiting examples of such methods include imaging methods (e.g., computed tomography (CT), magnetic resonance imaging (MRI), ultrasound, X-ray imaging, PET scans, radionuclide scans, bone scans), visual methods (e.g., through brain surgery), blood or biopsy tests (e.g., detection of EGFRvIII, glioblastoma cells often contain this mutation), histopathology, cytology, and flow cytometry.

In some embodiments, the bulk tumor size can be measured by assessments based on the size of tumor lesions determined from imaging methods. In specific embodiments, the assessments are performed in accordance with the Response Evaluation Criteria In Solid Tumors (RECIST) Guidelines, which are set forth in Therasse, P. et al., “New Guidelines to Evaluate the Response to Treatment in Solid Tumors,” J. of the Nat. Canc. Inst. 92(3), 205-216 (2000). For instance, in specific embodiments, lesions in the subject that are representative of bulk tumor size are selected so that they are at least ≧20 mm in their longest diameter at baseline (prior to treatment) when conventional imaging techniques are used (e.g., conventional CT scan, PET scan, bone scan, MRI or x-ray) and lesions that are at least ≧10 mm in their longest diameter at baseline should be selected when spiral CT scanning is used.

The invention provides a method of preventing, reducing, treating, or eliminating brain tumors, the method comprising administering to a subject in need thereof a therapeutically and/or prophylactically effective amount or regimen of sodium meta arsenite, the method comprising administering sodium meta arsenite to the subject at doses equal to or less than the maximum tolerated dose (MTD) or equal to or less than the no observed adverse effect level (NOAEL). The MTDs of sodium meta arsenite is typically based on the results of Phase I dose escalation trials.

The NOAEL, as determined in animal studies, is often used determining the maximum recommended starting dose for human clinical trials. The NOAELs can be extrapolated to determine human equivalent dosages (HEDs). Typically, such extrapolations between species are conducted based on the doses that are normalized to body surface area (i.e., mg/m²). In specific embodiments, the NOAELs are determined in either mice, hamsters, rats, ferrets, guinea pigs, rabbits, dogs, primates, primates (monkeys, marmosets, squirrel monkeys, baboons), micropigs and minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005. Accordingly, in certain embodiments, the regimen comprises administering a therapy at a dose less than the HED. For instance, the invention provides a method of preventing recurrence of brain tumors in a subject in remission, the method comprising administering to a subject in need thereof a prophylactically or therapeutically effective amount or regimen of sodium meta arsenite, the method comprising administering sodium meta arsenite to the subject at dose equal to or less than the HED.

### 3. Target Populations

In accordance with the invention, a prophylactically and/or therapeutically effective amount or regimen of sodium meta arsenite is administered to subjects with or at risk of developing a brain tumor, described above. Subjects at risk may be those with a genetic predisposition for a particular type of brain tumor. In one embodiment, a therapeutically effective amount or regimen sodium meta arsenite is administered to a subject that is undergoing or has undergone surgery to remove a brain tumor or neoplasm. In a specific embodiment, a therapeutically effective amount or regimen sodium meta arsenite is administered to a subject concurrently or following surgery to remove a brain tumor or neoplasm. In another embodiment, a therapeutically effective amount or regimen sodium meta arsenite is administered to a subject before surgery to remove a brain tumor or neoplasm and in some embodiments, sodium meta arsenite is administered during and/or after surgery.

In a specific embodiment, a therapeutically effective amount or regimen sodium meta arsenite is administered to subjects that will, are or have undergone radiation therapy. Among these subjects are those that have received chemotherapy, hormonal therapy and/or biological therapy including immunotherapy as well as those who have undergone surgery. Alternatively, a dosage regimen of sodium meta arsenite may be administered to the patient prior to or during the same time period in which the patient is undergoing chemotherapy with a non-arsenic based drug, or radiation therapy.

In certain embodiments, a therapeutically effective amount or regimen of sodium meta arsenite is administered to a subject who has failed or is refractory to one or more brain tumor therapies. In one embodiment, that a brain tumor is refractory to a therapy means that at least some significant portion of the brain cancer cells are not killed or that cancer cells division is not arrested. The determination of whether the brain cancer cells are refractory can be made either in vivo or in vitro by any method known in the art for assaying the effect of a therapy on cancer cells, using the art-accepted meanings of “refractory” in such a context.

### 4. Dosage & Frequency of Administration of Sodium Meta Arsenite

In one embodiment, the daily dosage of sodium meta arsenite administered to a subject to prevent, treat, eliminate, and/or manage brain tumor in a subject is 500 mg/kg or less, preferably 250 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 5 mg/kg or less, 2.5 mg/kg or less, 2 mg/kg or less, 1.5 mg/kg or less, or 1 mg/kg or less of a patient's body weight. The daily dosage may be administered as a single dosage or as multiple dosages throughout the day.

In another embodiment, the daily dosage of sodium meta arsenite administered to a subject to prevent, treat, eliminate, and/or manage brain tumor in a subject is a unit dose of 0.001 mg to 20 mg, 0.01 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. In certain embodiments the daily dosage of sodium meta arsenite administered to a patient is about 2.5 to 15 mg/day. The daily dosage may be increased or decreased during the treatment period, taking into account the patient's overall health, response

In certain embodiments, the daily dosage of sodium meta arsenite administered to a subject to prevent, treat, eliminate, and/or manage brain tumor in a subject is in the range of 0.01 to 10 g/m², and more typically, in the range of 0.1 g/m m² to 7.5 g/m², of the subject's body weight. In one embodiment, the dosage administered to a subject is in the range of 0.5 g/m² to 5 g/m², or 1 g/m² to 5 g/m² of the subject's body's surface area.

In certain embodiments of the invention, a daily dosage of sodium meta arsenite is administered to the patient on consecutive days, such as for three to twenty one consecutive days, although the total number of days of treatment may vary from patient to patient. In other embodiments, sodium meta arsenite is administered for a period of time, e.g., three days, followed by a period of time in which the patient is not treated with sodium meta arsenite, e.g., three days. Treatment may be repeated using the same pattern of treatment or a different pattern of treatment. In other embodiments, the patient may be treated with another anti-cancer agent, such as radiation therapy or chemotherapy during the periods when sodium meta arsenite is not administered; in those embodiments, the patient does not necessarily receive treatment every day.

Treatment with sodium meta arsenite may be carried as long as necessary to reduce or eliminate the brain tumor. Treatment may be as short as three days, for example and may continue for up to six months or longer. For example, treatment with sodium meta arsenite may be carried out for three days, such as three consecutive days, and up to three months or longer, although during the longer period the patient need not necessarily receive treatment every day.

In some embodiments, the prophylactically and/or therapeutically effective amount or regimen of sodium meta arsenite is administered in combination with one or more additional therapies, such as radiation treatment, chemotherapeutic agents, and/or with chemosensitizers. Preferably, the dosages of the one or more additional therapies used in the combination therapy is lower than those which have been or are currently being used to prevent, treat, and/or manage cancer. The recommended dosages of the one or more additional therapies currently used for the prevention, treatment, and/or management of cancer can be obtained from any reference in the art including, but not limited to, Hardman et al., eds., Goodman & Gilman's The Pharmacological Basis Of Basis Of Therapeutics, 10th ed, Mc-Graw-Hill, New York, 2001; Physician's Desk Reference (60.sup.th ed., 2006), which are incorporated herein by reference in its entirety. Typical chemotherapeutic agents that may be used in the practice of the present invention include for example alkylating agents, antifolates and topoisomerase inhibitors.

Examples of additional cancer therapeutic agents include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracyclin; anthramycin; asparaginase; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine (Ara-C); dacarbazine; dactinomycin; daunorubicin hydrochloride; decitabine (Dacogen); demethylation agents; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; histone deacetylase inhibitors (HDAC-Is); hydroxyurea; idarubicin hydrochloride; ifosfamide; ilmofosine; imatinib mesylate (Gleevec, Glivec); interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; lenalidomide (Revlimid); letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

The combination therapy can be administered in cycles such as where a therapeutic agent is administered on day one, followed by a second on day two, then a period without administration, followed by re-administration of the therapeutics on different successive days, is comprehended within the present invention. The dosage regimen will be determined by the patient's physician taking into account such factors as the patient's overall health, age, weight, response to treatment, and other relevant factors.

Sodium meta arsenite and the one or more additional therapies can be administered separately, simultaneously, or sequentially. The combination of agents may be administered to a subject by the same or different routes of administration. In alternative embodiments, two or more prophylactic or therapeutic agents are administered in a single composition. The combination therapy can also be administered in cycles such as where a therapeutic agent or treatment is administered on day one, followed by a second on day two, then a period without administration, followed by re-administration of the therapeutics on different successive days, is comprehended within the present invention.

Kits containing dosage units of sodium meta arsenite, formulated for oral or intravenous administration, are contemplated by the invention. The kits may contain sodium meta arsenite as the sole anti-brain tumor agent or may also contain other agents for treating brain tumors, such as an alkylating agent, antifolate or topoisomerase, formulated for delivery as appropriate for the specific agent, as well as a chemosentizing agent, if appropriate. The kit may contain sufficient amounts of the treatment agents for one or several rounds of treatment.

All publications, patents and patent applications mentioned in this specification are herein incorporated by reference into the specification to the same extent as if each individual publication, patent or patent application was specifically and individually indicated to be incorporated herein by reference. Citation or discussion of a reference herein shall not be construed as an admission that such is prior art to the present invention.

### 5. Pharmaceutical Compositions and Dosage Forms of Sodium Meta Arsenite

Pharmaceutical compositions and dosage forms of the invention comprise sodium meta arsenite, solvate, hydrate, stereoisomer, clathrate, or prodrug thereof. Pharmaceutical compositions and dosage forms of the invention can further comprise one or more excipients.

Pharmaceutical compositions and dosage forms of the invention can also comprise one or more additional active ingredients. Consequently, pharmaceutical compositions and dosage forms of the invention comprise sodium meta arsenite and optionally a second active agent (as in combination therapies described above).

Single unit dosage forms of the invention are suitable for oral and parenteral (e.g., subcutaneous, intravenous, bolus injection, intramuscular, or intraarterial), transdermal or transcutaneous administration to a subject. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a subject.

The composition, shape, and type of dosage forms of the invention will typically vary depending on their use. For example, a dosage form used in the treatment of an aggressive brain tumor may contain larger amounts of sodium meta arsenite and optionally one or more of other active ingredients it comprises than a dosage form used in the treatment of a less aggressive brain tumor. Similarly, a parenteral dosage form may contain smaller amounts of sodium meta arsenite and optionally one or more of other active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms of sodium meta arsenite comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

The compositions of the invention can comprise excipients that are well known in the art and are listed, for example, in the U.S. Pharmacopeia (USP) 25-NF20 (2002). In general, compositions of the invention comprise one or more active ingredients, a binder/filler, and a lubricant in pharmaceutically compatible and pharmaceutically acceptable amounts. In certain embodiment, dosage forms comprise sodium metal arsenite and optionally one or more other active ingredients, microcrystalline cellulose, pre-gelatinized starch, and magnesium stearate.

### 5.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining sodium meta arsenite in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, PA.), and mixtures thereof. An specific binder is a mixture of microcrystalline cellulose and sodium carboxymethyl cellulose sold as AVICEL RC-581

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

### 5.2 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

The following materials and methods were used in the examples described herein.

Vehicle: The test article, sodium meta arsenite, was formulated for PO administration in sterile water and for IV administration in saline.

Dose Formulation and Analysis: Sodium meta arsenite was formulated by MDSPS approximately 2 to 3 hours prior to administration. Dosing solutions were prepared by dissolution/suspension of the test article in sterile water to achieve a nominal concentration of 1.0 mg/mL for the oral administration and in saline at a nominal concentration of 0.5 mg/mL for the IV administration. Clear solutions were obtained. Sodium meta arsenite formulation information is listed in Table 2.

Seventy-three (plus ten spares) male CD-1 (ICR) albino mice (approximately 4 weeks old, 18-27 g upon arrival) were obtained from Charles River Canada, Inc. Upon receipt, a health inspection was performed on each animal. This inspection included evaluation of animal coat, extremities, and orifices, along with any abnormal posture or movement. Following successful inspection, animals were assigned identification numbers, randomized by weight, and allocated to treatment groups (see Table 3). Animals were fasted for 2 hours prior to dosing and weighed in the morning, prior to dosing (21-29 g). Dose calculations were made based on the most recent animal body weights. All animals were dosed PO at 10 mg/kg or IV at 5 mg/kg, with a target dose volume of 10 mL/kg. Animals were observed regularly following drug administration, and during subsequent serial blood collection. Following administration, blood samples (0.2 to 0.3 mL) were obtained via the vena cava under isoflurane anesthesia at 5, 15, 30, 60, 120, 240, 480, 1440, 1920, 2880, 3360 and 4320 min post-dose. Immediately following each blood collection, the animals will be sacrificed and the brain collected at 5, 30, 60, 120, 240, 480, 1440, 2880 and 4320 min post-dose. Blood samples were transferred into tubes containing K2-EDTA as the anticoagulant, and immediately placed on ice pending centrifugation (3200 g for 10 min refrigerated). The plasma samples were transferred into pre-labeled microfuge tubes. Microfuge tubes were capped and placed on dry ice until transferred to frozen storage. Brains were collected and transferred into appropriately labeled, 30 mL tubes and placed on ice pending frozen storage. All animals were sacrificed and discarded following collection of the last PK sample. All PK samples (plasma and brain) were stored frozen pending concentration assessment by ICP-MS.

### Example 1

### Determination of Plasma and Brain Pharmacokinetic Profile of Sodium Meta Arsenite in Mice

Analysis: The plasma and whole brain samples were digested with concentrated nitric acid in a Teflon bomb at 105° C. The digestate was diluted to 40 mL for analysis by ICP-MS. The digestate was aspirated into the inductively coupled plasma and the resulting ions were extracted by a vacuum interface into a quadrapole mass analyzer. The amount of arsenic in the samples was measured by comparison of the response of a standard solution at mass 75. NRCC DOLT-3 and DORM-2 were analyzed as standard reference materials.

Pharmacokinetic (PK) Analysis: The drug concentration versus time data were analyzed to generate the following PK parameters by noncompartmental analysis (Table 1):

TABLE 1

| Parameter | Units | Notes |
|---|---|---|
| Tmax | hour | Time to reach maximum concentration |
| Cmax | ng/mL | Highest concentration within the timeframe |
| AUCₐₗₗ | ng * h/mL | Area under the curve, generated by log-linear |
| | | trapezoidal method for Interpolation |
| F | % | Bioavailability of orally dosed animals |

No clinical signs were observed during testing. There were no significant time deviations (greater than 2 minutes) from the theoretical sample collection time points. The concentrations of sodium meta arsenite in biological matrix samples were obtain (raw data not shown). Briefly, the concentration of sodium meta arsenite was measured in plasma of animals with ID numbers 1001-1036 treated with 10 mg/kg of sodium meta arsenite by way of PO (oral); the concentration of sodium meta arsenite was measured in plasma of animals with ID numbers 2001-2037 treated with 4 mg/kg of sodium meta arsenite by way of IV (injection); the concentration of sodium meta arsenite was measured in brain of animals with ID numbers 1001-1036 treated with 10 mg/kg of sodium meta arsenite by way of PO; and the concentration of sodium meta arsenite was measured in brain of animals with ID numbers 2001-2037 treated with 5 mg/kg of sodium meta arsenite by way of IV. The sodium meta arsenite concentration measurement data are summarized in Tables 4-7. No anomalies associated with the analysis of sodium meta arsenite were observed. Pharmacokinetic (PK) analysis was completed following PO and IV administration (see Tables 4-7).

TABLE 2
Sodium meta arsenite Formulation Information

| | | Volume | |
|---|---|---|---|
| Test Article | Amount weighed | Prepared | Formulation |
| Concentration | (mg) | (mL) | Appearance |
| Sodium Meta- | 15.02 | 15 | Clear solution |
| Arsenite | | | |
| (1 mg/mL) | | | |
| Sodium Meta- | 7.38 | 15 | Clear solution |
| Arsenite | | | |
| (0.5 mg/mL) | | | |

TABLE 3
Treatment Groups

| | | | | Dose | | Brain | |
|---|---|---|---|---|---|---|---|
| | Test Article | | Dose | volume | Plasma PK Sample | PK | |
| Group | (vehicle) | Route | (mg/kg) | (mL/kg) | Times (min) | (min) | Animal No. |
| 1 | Sodium meta- | PO | 10 | 10 | 5 | 5 | 1001-1036 |
| | Arsenite | | | | 15 | 30 | |
| | (Water) | | | | 30 | 60 | |
| | | | | | 60 | 120 | |
| | | | | | 120 | 240 | |
| | | | | | 240 | 480 | |
| | | | | | 480 | 1440 | |
| | | | | | 1440 | 2880 | |
| | | | | | 1920 | 4320 | |
| | | | | | 2880 | | |
| | | | | | 3360 | | |
| | | | | | 4320 | | |
| 2 | Sodium meta- | IV | 5 | 10 | 0¹ | 0¹ | 2001-2036 |
| | Arsenite | | | | 5 | 5 | |
| | (Saline) | | | | 15 | 30 | |
| | | | | | 30 | 60 | |
| | | | | | 60 | 120 | |
| | | | | | 120 | 240 | |
| | | | | | 240 | 480 | |
| | | | | | 480 | 1440 | |
| | | | | | 1440 | 2880 | |
| | | | | | 1920 | 4320 | |
| | | | | | 2880 | | |
| | | | | | 3360 | | |
| | | | | | 4320 | | |

¹Samples obtained from a single animal only (animal no. 2037)

TABLE 4
Plasma PK Results - Group 1: Sodium meta-Arsenite (PO, 10 mg/kg)

| Pharmacokinetic (PK) | | PK Results |
|---|---|---|
| Parameter | Units | (N = 3)¹ |
| AUCa11 | Mg * hr/Kg | 6.85 |
| Cmax | mg/Kg | 0.52 |
| Tmax | hr | 4 |
| F % | % | 101% |

¹Pharmacokinetic analysis completed using the mean (N = 3) concentration value at each

TABLE 5
Plasma PK Results - Group 2: Sodium meta-Arsenite (IV, 5 mg/kg)

| Pharmacokinetic (PK) | | PK Results |
|---|---|---|
| Parameter | Units | (N = 3)¹ |
| AUCaII | mg * hr/Kg | 3.38 |
| Cmax | mg/Kg | 0.41 |
| Tmax | hr | 0.25 |
| F % | % | N/A² |

¹Pharmacokinetic analysis completed using the mean (N = 3) concentration value at each collection time point
²N/A = Not applicable

TABLE 6
Brain PK Results - Group 1: Sodium meta-Arsenite (PO, 10 mg/kg)

| Pharmacokinetic (PK) | | PK Results |
|---|---|---|
| Parameter | Units | (N = 3)¹ |
| AUCa11 | mg * hr/Kg | 6.73 |
| Cmax | mg/Kg | 0.33 |
| Tmax | hr | 8 |
| F % | % | 87% |

¹Pharmacokinetic analysis completed using the mean (N = 3) concentration value at each

TABLE 7
Brain PK Results - Group 2: Sodium meta-Arsenite (IV, 5 mg/kg)

| Pharmacokinetic (PK) | | PK Results |
|---|---|---|
| Parameter | Units | (N = 3)¹ |
| AUCaII | mg * hr/Kg | 3.86 |
| Cmax | mg/Kg | 0.32 |
| Tmax | hr | 4 |
| F % | % | N/A² |

¹Pharmacokinetic analysis completed using the mean (N = 3) concentration value at each collection time point
²N/A = Not applicable

CONCLUSIONS: No adverse effects were observed following oral and IV administration of sodium meta arsenite. No significant deviations from the nominal sample collection bleed times occurred. No anomalies associated with the analysis of the study samples were observed. Pharmacokinetic (PK) analysis was completed for all concentrations tested.

### Example 2

### Effects of Sodium Meta Arsenite on Cell Survivability

The purpose of this study was to study the effect of sodium meta arsenite concentration on cell survivability.

A stock solution of sodium meta arsenite was prepared by dissolving 5 mM sodium meta arsenite in 1 N NaOH (more than ×1000). Culture medium was prepared by DMEM supplemented with 10% FBS. Washing medium used was DPBS supplemented with 2% FBS. Cell types that were tested were Human caner cell line (MDA-MB-231; (breast cancer); u87MG (brain cancer). Other reagents used were Hoechst 33342 (bixbenzimide), PI (propidium iodide) and Trypsin-EDTA.

Frozen cells were thawed at 37° C. and 2×10⁶ cells per vial were washed with DPBS and DMEM. The cells were seeded in a 25T flask with 5 ml DMEM and cultured for 3 days. Three days later, cells are treated with trypsin-EDTA to recover the attached cells. 2×10⁴ cells were seeded in a 24 well plate. 2-3 days later, when cells showed 70-80% confluency, the cells were treated with sodium meta arsenite, 3 wells per concentration of 0.005, 0.01, 0.03, 0.05, 0.075, 0.1 uM. The remaining 6 wells were controls. Cells were treated with sodium meta arsenite for 24 hours. See Table 8. After 24 hours of treatment, cells were trypsinized with trypsin-EDTA, washed, and then stained with 10 ug/ml Hoechst 33342 (bisbenzimide) and 10 ug/ml propidium to check for survivability under the fluorescence microscope. Under the fluorescence microscope, Hoechst 33342-stained blue cells indicate live cells, and propidium iodide-stained red cells indicate the dead cells. Survivability was calculated as follows, and mean+/−SD was calculated by calculating the repeated number.

Survivability=Hoechst staining cells (blue)/Hoechst+propidium iodide staining cells (red)

*Hoechst has permeability into the nucleus of both live and dead cells, but propidium iodide can only permeate the nucleus of dead cells.

TABLE 8
24 well plate cell treatment

| | | | | | |
|---|---|---|---|---|---|
| 0.005 uM | 0.01 | 0.03 | 0.05 | 0.075 | 0.1 |
| 0.005 | 0.01 | 0.03 | 0.05 | 0.075 | 0.1 |
| 0.005 | 0.01 | 0.03 | 0.05 | 0.075 | 0.1 |
| Control | Control | Control | Control | Control | Control |

TABLE 9
Cell survivability (%) of human MDA-MB-231 and u87MG cells

| Sodium meta arsenite | Breast cancer | Brain tumor |
|---|---|---|
| concentration | (MDA-MB-231) | (u98MG) |
| 0.1 uM | 15 +/− 3 | 5 +/− 2 |
| 0.075 uM | 20 +/− 5 | 10 +/− 3 |
| 0.05 uM | 30 +/− 4 | 20 +/− 5 |
| 0.03 uM | 70 +/− 4 | 50 +/− 6 |
| 0.01 uM | 75 +/− 5 | 60 +/− 7 |
| 0.005 uM | 85 +/− 5 | 70 +/− 7 |

* 6 replicates

Conclusion: The in vitro study shows that sodium meta arsenite showed receptivity in breast cancer cells (MDA-MB-231) and brain tumor cells (u98MG). The breast cancer cells at 0.03-0.05 uM sodium meta arsenite and brain tumor cells at 0.03 uM sodium meta arsenite showed LD50. There was no significant difference between the two cell lines regarding survivability at the above range of sodium meta arsenite concentrations, but brain tumor cells showed more receptivity. See Table 9.

### Example 3

### Summary of Human Patient Reports on the Effect on Sodium Meta Arsenite on Brain Tumors

The following information was gathered from brain tumor patients treated with an oral dosage form of sodium meta arsenite, formulated at 2.5 mg sodium meta arsenite/pill.

TABLE 10

| | | | Dosage of | |
|---|---|---|---|---|
| | | | sodium meta | |
| | Personal | | arsenite | |
| | Information | Symptoms | Administration | Effect of Sodium meta arsenite |
| 1 | Pt # 1 | 1. Pt # 1 suffered from | 1 pill taken 30 | Pt # 1 did not find any particular |
| | Age: 57 | worsening headaches | minutes before | side effects, and within 5 days her |
| | Gender: | since 2003, and in 2005 | meal | pain lessened, her hair began to |
| | Female | August, Pt # 1 was | | grow back, her facial tone became |
| | | diagnosed with brain | | healthier, her head felt clearer, and |
| | | tumor with a tumor size | | the pain eventually disappeared. |
| | | of 3 cm, at stage III. | | After 15 days of sodium meta |
| | | The tumor was located | | arsenite consumption, Pt#1's scans |
| | | on the central top of the | | looked much better according to the |
| | | brain and surgery was | | attending physician, and after 75 |
| | | impossible. | | days of sodium meta arsenite |
| | | 2. Due to the | | treatment her MRI showed that her |
| | | impossibity of surgery, | | brain tumor was completely gone. |
| | | Pt # 1 began | | Pt # 1 was not on any other |
| | | conventional drug | | medication during sodium meta |
| | | treatment | | arsenite treatment. |
| | | recommended by the | | |
| | | hospital. Thereafter, Pt | | |
| | | # 1 had severe | | |
| | | headaches and eye | | |
| | | pains; she could not | | |
| | | sleep at night, and had | | |
| | | to wear a hat because | | |
| | | her head felt cold. | | |
| | | Pt # 1 also noticed she | | |
| | | was losing a lot of hair. | | |
| 2 | Pt. # 2 | 1. Pt # 2 was diagnosed | Pt #2 started | After taking sodium meta arsenite, |
| | Age: 67 | with lung cancer stage | taking sodium | the tumor in the brain disappeared |
| | Gender: | IV and metastatic | meta arsenite | and for the lung cancer, the |
| | Male | tumor in the left frontal | after the brain | conditions appear to be stable. |
| | | lobe of the brain. | surgery, and | |
| | | 2. In the left temporal | took four (4) | |
| | | region, Pt # 2 had a | pills/day; 2 in | |
| | | calcified mass and | the morning and | |
| | | possibly calcified | 2 in the evening. | |
| | | meningioma in the | | |
| | | right temporal lobe. | | |
| | | 3. Pt. # 2 had brain | | |
| | | surgery. | | |

The above anecdotal information provides further evidence that sodium meta arsenite is an effective treatment for primary and secondary brain tumors.

### Example 4

### In Vitro Cytotoxic Effects of Sodium Meta Arsenite in Glioblastoma Cell Line

U-87 MG cells, U373 cells, T98G (human glioblastoma cells) or U373 neuroblastoma cells were injected intracranially into the brains of test mice that were then treated with various amounts of sodium meta arsenite. The cytotoxic effects of the treatment were evaluated by cell counting using a commercially available Cell Counting Kit-8 (CCK-8; Dojindo, Japan) or by an MTT cell proliferation assay (Sigma, USA) or by ELISA reader system.

TABLE 11
Treatment groups (1)

| Group | | | Treatment | N |
|---|---|---|---|---|
| I (Control) | U-87MG | 1 week | Control(5% Glucose of | 7 |
| | Cell | later | DDW, P.O.), | |
| | I.C. | | Normal saline (I.P.) | |
| II (Sodium meta | injection | | Sodium meta arsenite | 7 |
| arsenite 1.25 mg/kg) | (2 × 10⁵/ | | 1.25 mg/kg, P.O. | |
| III (sodium meta | 5 μl) | | Sodium meta arsenite | 7 |
| arsenite 2.5 mg/kg) | | | 2.5 mg/kg, P.O. | |
| IV (sodium meta | | | Sodium meta arsenite | 7 |
| arsenite 5 mg/kg) | | | 5 mg/kg, P.O. | |

TABLE 12
Treatment groups (2)

| Group | | | Treatment | N |
|---|---|---|---|---|
| I (Control) | U-87MG | 1 day later | Control (5% Glucose | 7 |
| | Cell | | of DDW, P.O.), | |
| | I.C. | | Normal saline (I.P.) | |
| II (Sodium meta | injection | | Sodium meta arsenite | 7 |
| arsenite 2.5 mg/kg) | (2 × 10⁵/ | | 2.5 mg/kg, P.O. | |
| III (Sodium meta | 5 μl) | | Sodium meta arsenite | 7 |
| arsenite 5 mg/kg) | | | 5 mg/kg, P.O. | |
| IV (Sodium meta | | | Sodium meta arsenite | 7 |
| arsenite 10 mg/kg) | | | 10 mg/kg, P.O. | |

TABLE 13
MRI image check group

| Group | Treatment | N |
|---|---|---|
| Control | Control(5% Glucose of DDW, P.O.), | 3 |
| | Normal saline (I.P.) | |
| Sodium meta arsenite 5 mg/kg | Sodium Meta arsenite 5 mg/kg, | 3 |
| | P.O. | |

### 2) Measurement of Tumor Mass (Hematoxylin and Eosin Staining)

Tumor mass (mm³)=Tumor length (mm)×Tumor width (mm)²×0.5

### 3) Blood Biochemical Tests

Aspartate transaminase (AST), alanine transaminase (ALT) Sigma Diagnostics Kit UVIKON, Kontron Inc.

### Results: In Vitro Cytotoxic Effects of Sodium Meta Arsenite in Glioblastoma Cell Lines

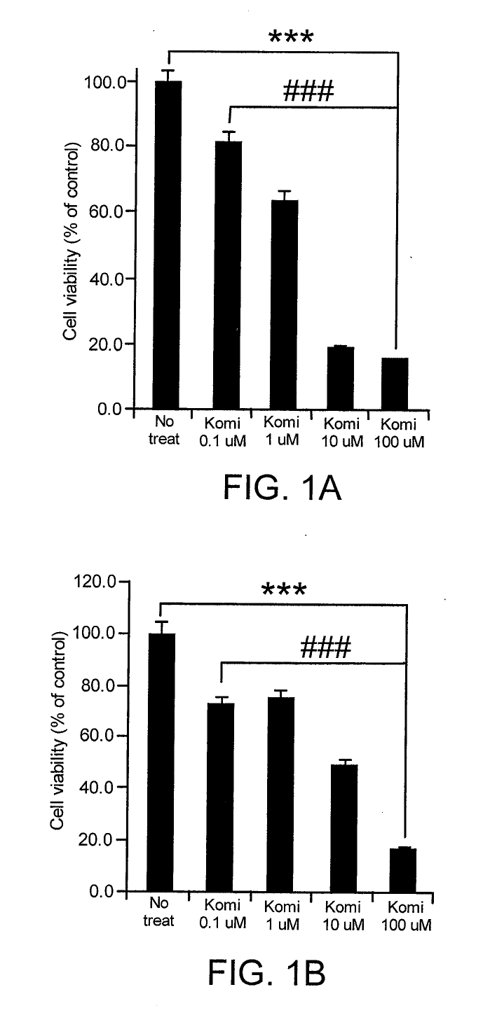

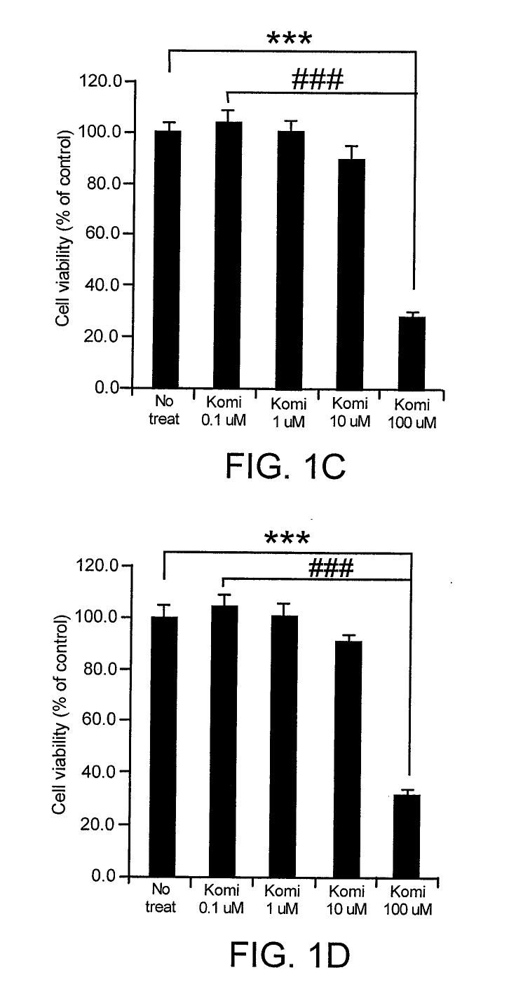

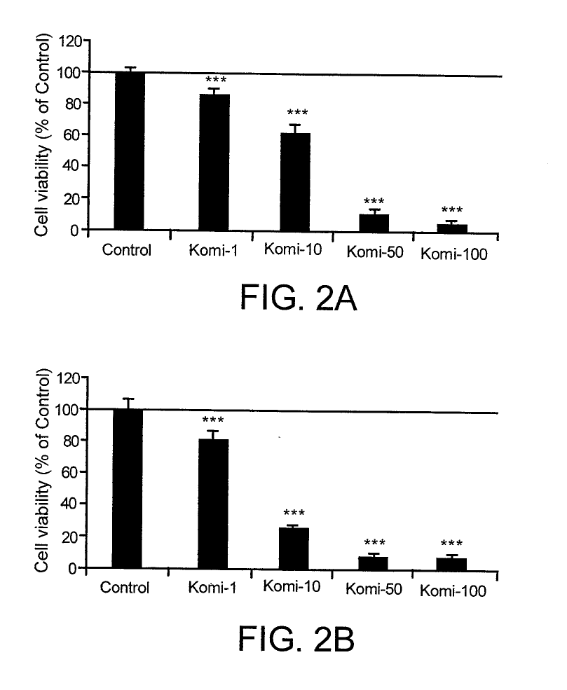

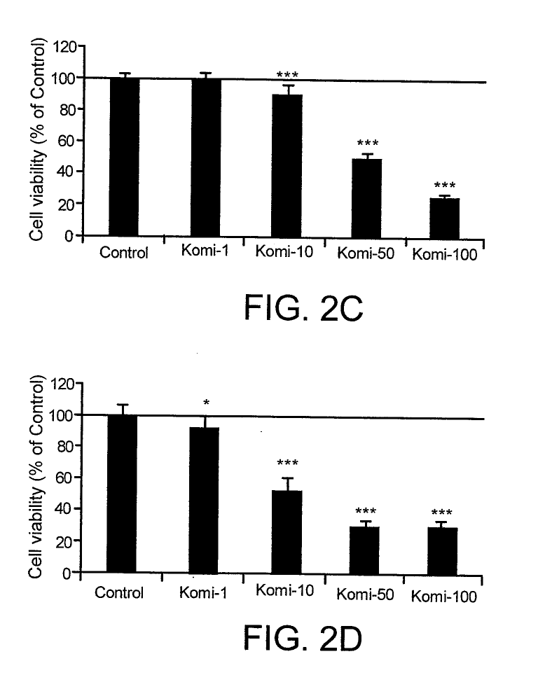

Sodium meta arsenite showed significant anticancer effects in all four types of neuroblastoma cell lines (U-87MG, U373, T98G, U373) (***p<0.001 compared with control group) (FIG. 1A-1D).

Sodium meta arsenite showed dose-dependent anticancer effects in U-87MG, U373, T98G and U373 neuroblastoma cell lines when treated at 1, 10, 50, 100 μM.

### Efficacy Study of Sodium Meta Arsenite in U-87MG Orthotopic Glioblastoma Model

(1) Efficacy of Sodium Meta Arsenite in Primary Orthotopic Brain Tumor Model I

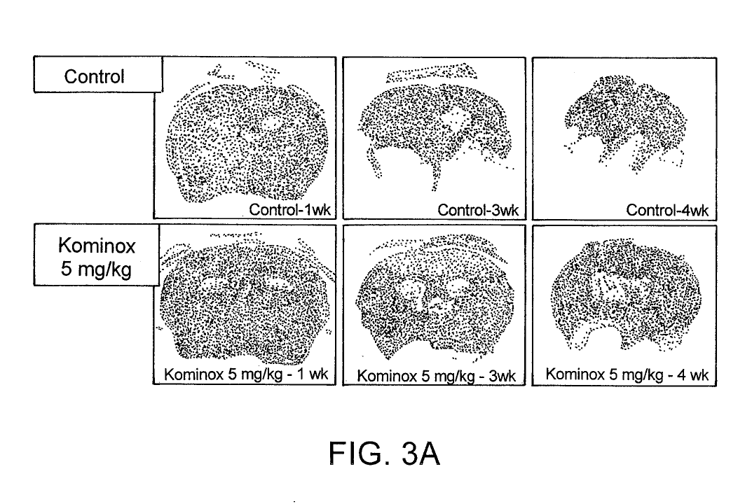

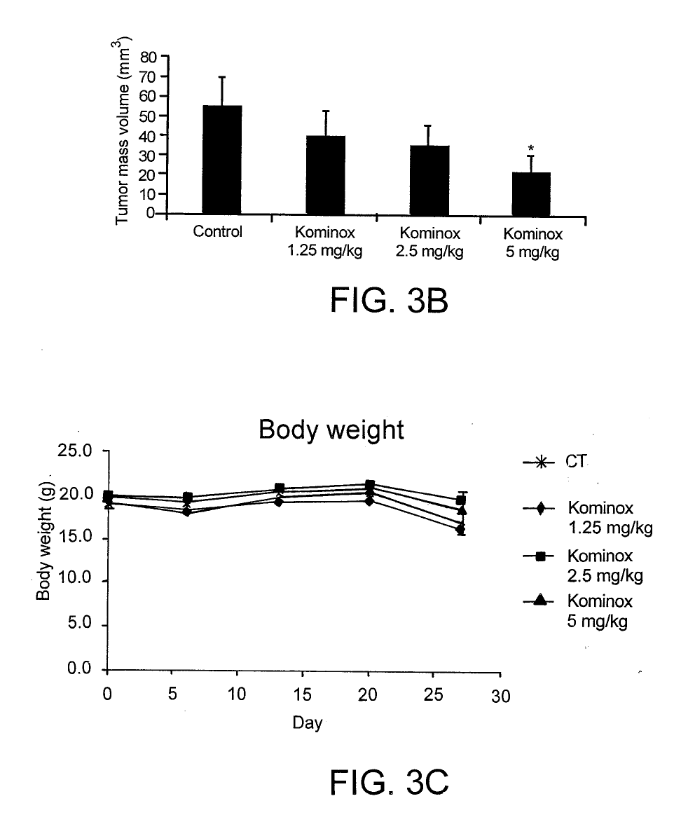

None of the treatments caused any side effects in terms of liver enzyme levels, body weight and lethality. Tumor mass of the control group was 55.4±13.5 mm³. The administration of 1.25 mg/kg, 2.5 mg/kg and 5 mg/kg of sodium meta arsenite caused 28% (40.2±12.3 mm³), 35% (36.2±9.2 mm³) and 60% (21.9±7.7 mm³) inhibition of tumor growth, respectively. (FIG. 3A). MRI imaging showed a decrease of tumor mass in a group treated with 5 mg/kg of Sodium meta arsenite (FIG. 3B). There was no significant difference in body weight between groups (FIG. 3C).

(2) Efficacy of Sodium Meta Arsenite in Primary Orthotopic Brain Tumor Model II

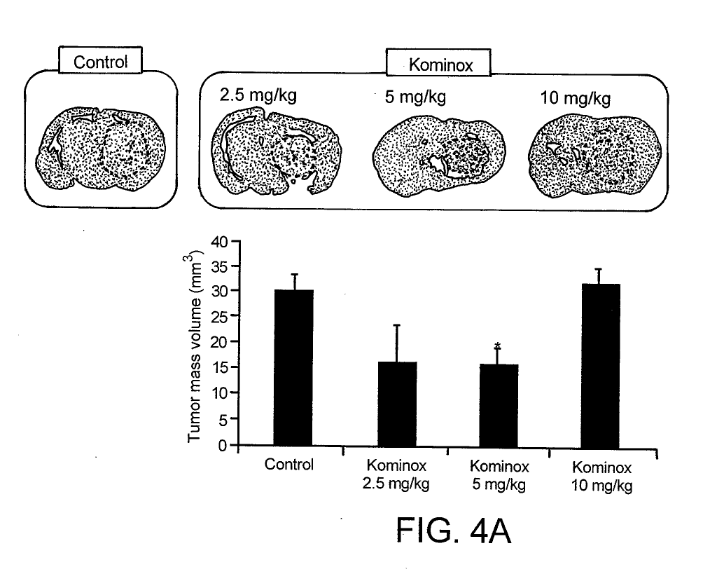

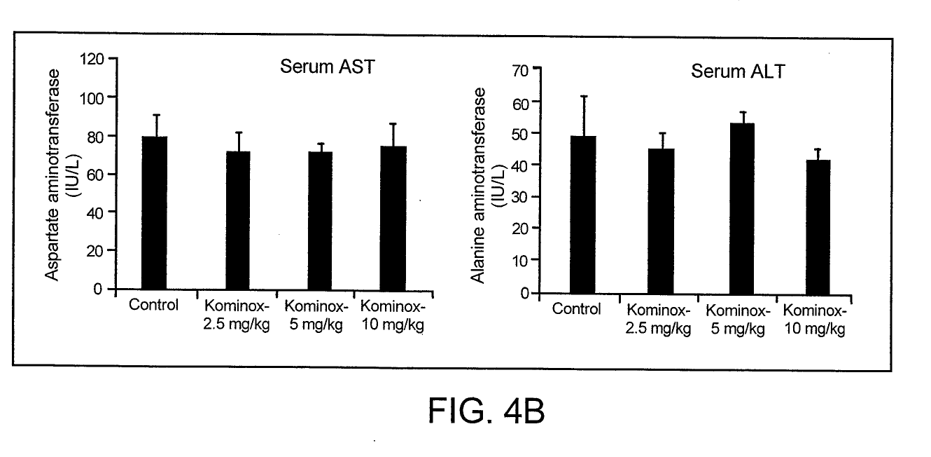

Tumor mass of the control group was 30.0±3.6 mm³. The administration of 2.5 mg/kg and 5 mg/kg sodium meta arsenite caused 45% (16.7±7.9 mm³) and 46% (16.3±3.0 mm³) inhibition of tumor growth, respectively. The administration of 10 mg/kg of sodium meta arsenite showed no anticancer effect. (FIG. 4A). There was no significant difference in serum ALT and AST between the groups (FIG. 4B).

What is claimed is:

1. A method of treating a brain tumor in a patient comprising administering to the patient a therapeutically effective amount of sodium meta arsenite.

2. The method of claim 1 wherein the therapeutically effective amount is in the range of from about 2.5 mg/day to 10 mg/day.

3. The method of claim 1 wherein the therapeutically effective amount of about 5 mg/day.

4. The method of claim 1 wherein the sodium meta arsenite is administered orally.

5. The method of claim 2 wherein the sodium meta arsenite is administered daily for a period of from about three days to about three months.

6. The method of claim 1 wherein the patient was treated with a chemotherapeutic agent prior to administration of the sodium meta arsenite and the brain tumor is refractive to the chemotherapeutic agent.

7. The method of claim 1 wherein the brain tumor is selected from the group of brain tumors consisting of oligodendroglioma, oligoastrocytoma, astrocytoma, medullablastoma, meningioma, Schwannoma, hemangioblastoma and hemangiocytoma.

8. The method of claim 1 wherein the patient is treated with at least one anti-brain tumor medicament in addition to and different from sodium meta arsenite.

9. The method of claim 1 wherein the patient underwent surgery to remove all or part of the brain tumor prior to administration of sodium meta arsenite.

10. The method of claim 1 wherein the patient receives radiation therapy to treat the brain tumor prior to or following the administration of sodium meta arsenite.

11. The method of claim 8 wherein the at least one anti-brain tumor medicament is a chemotherapeutic agent or chemosensitizer.

12. The method of claim 11 wherein the chemotherapeutic agent is administered after completion of a dosage regimen of sodium meta arsenite, during a dosage regimen of sodium meta arsenite or prior to a dosage regimen of sodium meta arsenite.

13. The method of claim 12 wherein the chemotherapeutic agent is administered in combination with a chemosensitizer.

14. The method of claim 1 further comprising the step of monitoring the brain tumor for change in size.
